(19) United States
(12) Patent Application Publication
(10) Pub. No.: US 2010/0105983 A1
(43) Pub. Date: Apr. 29, 2010

(54) SYSTEM AND METHOD FOR NAVIGATING A TOOL WITHIN A BODY CONDUIT

(75) Inventors: Katsumi Oneda, Alpine, NJ (US); Ron Hadani, Cresskill, NJ (US)

Correspondence Address: MARTIN D. MOYNIHAN d/b/a PRTSI, INC., P.O. BOX 16446, ARLINGTON, VA, 22215, US

(73) Assignee: Vision - Sciences Inc., Orangeburg, NY (US)

(21) Appl. No.: 12/525,110

(22) PCT Filed: Aug. 2, 2007
(86) PCT No.: PCT/IL2007/000969

§ 371 Date: Jul. 30, 2009

(30) Foreign Application Priority Data
Jan. 30, 2007 (IL) PCT/IL2007/000116

(51) Int. Cl. A61B 1/01

(52) U.S. Cl. 600/115

(57) ABSTRACT

Apparatus and method for navigating surgical tools within a body cavity. Embodiments of the invention are particularly useful for displacing a surgical tool within a body conduit such as an intestine. Embodiments comprise a balloon catheter having a shaft, and a surgical tool operable to slide along that shaft. In some embodiments the surgical tool comprises a second balloon catheter.

### RELATED APPLICATIONS

The present application draws priority from PCT Application IL2007/00116 filed Jan. 30, 2007, the contents of which are incorporated herein by reference.

### FIELD OF THE INVENTION

The present invention relates to devices and tools for navigating a tool within a body cavity. Embodiments of the invention include methods and devices for positioning surgical tools within a lumen of a body conduit such as an intestine.

### BACKGROUND OF THE INVENTION

Catheters, endoscopes, and minimally invasive probes of various sorts are used today for many sorts of minimally invasive surgical and diagnostic procedures. Directing a diagnostic tool or a treatment tool to an intervention site is often performed by advancing the device through a body conduit, often with benefit of visual guidance by means of an external imaging modality and/or a camera or optical arrangement on the inserted device. Insertable devices may be steerable, and an operator advances the device through the body conduit until it reaches an intervention site where diagnostic and/or treatment work is performed.

Advancing and retracting an endoscope or similar tool through a body conduit or other body cavity requires a certain level of skill and experience and in some circumstances can be a time-consuming performance. Prior art does not provide efficient methods enabling to use an endoscope or other tool to reach a desired site within such a body conduit, remove the endoscope, and then easily or automatically revisit the site.

### SUMMARY OF THE INVENTION

The present invention, in some embodiments thereof, comprises apparatus and methods for navigating a tool within a body cavity or body conduit such as an intestine. Embodiments of the invention enable simple and rapid navigation of tools within the body, and easy revisiting of a previously visited intervention site.

Various embodiments of the invention comprise a balloon catheter having a distal portion which comprises an expandable element such as an inflatable balloon, and a proximal shaft, and further comprise a surgical tool designed to slide along the catheter shaft. Methods for using the apparatus include advancing the catheter to a position within a body conduit, expanding the expandable element (e.g. by inflating a balloon of a balloon catheter) to fix the catheter in place, and then sliding a tool along the catheter shaft. In this manner, the catheter shaft, fixed in place within the conduit by expansion of the expandable element (e.g. by inflation of a balloon), fulfills the function of a guide wire which serves to guide an associated tool through the twists and turns of the body conduit, and enables rapid displacement of the tool within the conduit, and easy re-visiting of a previously visited site. Embodiments of the present invention thus provide means for guiding insertion, advancement, and retraction of a variety of surgical and diagnostic tools within body cavities and in particular body conduits.

Balloon catheters of the present invention generally comprise an inflation lumen within a shaft, through which a fluid may be supplied under pressure to inflate a distal balloon. The catheter shaft may comprise a guide wire external to other portions of the shaft, for facilitating connection to external tools and/or to attachment mechanisms as herein described. The catheters will generally be provided with a proximal connector for connecting the catheter to a balloon inflation fluid source. Those proximal connectors may be removable (e.g. a Tuohy-Borst connector or similar connector) to enable to insert the catheter shaft into a narrow conduit (such as a working channel of an endoscope) from the proximal end of the catheter. Catheters of embodiments of the invention may comprise additional lumens within their shafts, such as for example irrigation lumens for supplying irrigating fluid to an intervention site, for clearing an optical pathway to enable visual inspection of the site, or to supply a lubricating material. Catheters may comprise additional features, such as cameras or optical arrangements for providing imaging capabilities, and angling mechanisms for steering the catheter and directing the orientation of the catheter's distal head.

The surgical tool may be slidably connectable to the catheter shaft. For example, the shaft may be sized to slide through a lumen provided in the catheter shaft, or the catheter shaft may be sized to slide through a lumen in the tool. As an example of the latter construction, the tool may be an endoscope, and the catheter shaft may be sized to be slidably insertable into a working channel of that endoscope.

Alternatively, the apparatus may comprise an attachment mechanism providing a bridging attachment between catheter and surgical tool, the attachment mechanism providing a slidable connection to the catheter shaft and a slidable or fixed connection to the surgical tool. Tool and catheter may be manufactured to be permanently or semi-permanently connected and associated, or may be attachable one to another and detachable one from another at will. For example, the attachment mechanism may be formed as a sleeve slidably connectable to a catheter shaft and rigidly connectable to a tool such as an endoscope, using binding bands to ensure a rigid connection between endoscope and attachment mechanism. Alternatively, such a sleeve may be slidably connectable to both catheter and tool. Such a sleeve may comprise a general-purpose lumen through which an endoscope or other tool may be inserted and bound. The attachment channel may be comprised within a sheath which extends along part or all of an endoscope or other tool and having a protective function, sterilizing function, or other function along with its function as an attachment mechanism.

Optionally, catheter, attachment mechanism and optionally an endoscope may be prepared in matching dimensions and with paired fasteners operable to bind the parts together, and may be packaged and distributed as a kit. Such a kit may include a plurality of catheters of varying sizes, catheters with expandable features other than balloons, a selection of attachment mechanisms adaptable to various tool sizes and tool shapes, a selection of bushings for adapting to various tool sizes, etc.

In some embodiments of the apparatus the associated tool is an endoscope, and the catheter is either insertable through a working channel of the endoscope or insertable through an external sleeve or conduit connected or connectable to that endoscope. Such an external conduit is an embodiment of an “attachment mechanism” referred to above. In some embodiments the conduit is less than 20 cm long. In some embodiments the conduit is less than 10 cm long, and in some embodiments less then 2 cm long. In other embodiments the conduit may be 50 cm long or longer, and may even extend over most or all of the length of the attached endoscope or other tool. In some embodiments the conduit is formed as a disposable sheath into which an endoscope may be inserted. In some embodiments a plurality of such conduits or other forms of attachment mechanism are provided intermittently along the length of a tool such as an endoscope, to ensure that tool and catheter shaft remain in proximity along their lengths.

In some embodiments, the “tool” connectable to a first balloon catheter is a second balloon catheter. In these embodiments two balloon catheters are provided, each having an inflatable balloon on a distal portion of the catheter. The catheters may be linked together in ‘side-by-side’ fashion by use of one or preferably a plurality of attachment mechanisms, or alternatively one catheter may be slidably inserted within the other catheter. Utilizing such paired balloon catheters it is possible to easily advance the catheters through a body conduit such as an intestine by inserting a first catheter a first distance, inflating its balloon to fix its position within the body conduit, then advancing the second catheter by sliding the second catheter along the first catheter, then inflating the second balloon to immobilize the second catheter, deflating the first balloon to free the first catheter, and further advancing the first catheter by pushing it forward with respect to the immobilized second catheter.

In some embodiments the tool associated with the apparatus comprises distance markings on its proximal shaft, which markings enable to measure the distance by which the tool has been inserted into a body, and/or to measure a distance by which a first catheter has been advanced with respect to a second catheter, and/or to measure a distance of a tool operating tip from an anchoring balloon.

According to another aspect of the invention, methods are provided for using devices described herein during surgical interventions.

A method is presented for navigating a surgical tool within a body conduit, comprising inserting into a body conduit a catheter having a proximal shaft and a distal head which comprises an inflatable balloon, advancing the catheter to a desired location, inflating the balloon to fix the position of the catheter head at the desired position, and then navigating a surgical tool within the body conduit by sliding the tool along the catheter shaft. Optionally, fixing of position of the inflated balloon within a body conduit may be facilitated by inflating the balloon in a position where a narrowing of the conduit helps prevent the inflated balloon from being retracted. This method may be particularly useful for navigating a tool within an intestine, by inflating a balloon positioned just beyond the Ileo-Cecal valve. Positioning of the balloon catheter after the Ileo-Cecal valve may be accomplished by inserting the catheter through a working channel of an endoscope, advancing a distal portion of the endoscope through the intestine to the vicinity of the Ileo-Cecal valve, extending the distal balloon portion of the catheter through the Ileo-Cecal valve, inflating the balloon, and then partially or fully retracting the endoscope from the intestine, while leaving the balloon catheter in place with the balloon positioned at the Ileo-Cecal valve. Once the catheter is in place the endoscope may be advanced and retracted along the catheter and used for diagnostic and therapeutic purposes, or the endoscope may be entirely removed from the intestine and an additional surgical tool may be attached to the catheter shaft, either directly or using an attachment mechanism to slidably attach surgical tool to the catheter shaft. The attached tool can then be advanced and retracted along the catheter shaft and used to do diagnostic or therapeutic work. Alternatively, a tool can be advanced beyond the anchoring balloon catheter, for doing therapeutic or diagnostic work in the small intestine.

The shaft of the endoscope or other attached therapeutic tool can be marked with distance markings. The method may further include sliding the surgical tool along the shaft to a first position within the body conduit, recording that first position as indicated by distance markings on the surgical tool shaft, sliding the tool along the shaft to a second position, and returning the tool to the first position by sliding the tool along the shaft until distance markings on the tool shaft correspond to distance markings recorded when the tool was at the first position. This method may be particularly useful when excising a large growth, such as for example a large polyp within an intestine. Prior art methods comprise removal of excised tissue through the working channel of an endoscope, yet such a working channel is of limited diameter and allows only limited amounts of tissue to be removed at one time. Using embodiments here presented, a large growth may be excised, seized using a tool provided through a working channel of an endoscope, then the entire endoscope may be retracted from the body conduit, optionally after marking it's position according to proximal distance markings provided on the endoscope. Once the seized tissue is removed from the endoscope, the endoscope may be re-introduced into the body cavity, slid along the catheter shaft as described herein, and rapidly restored to the excision site for inspection, further excision, or for continuation of a systematic exploration of the conduit.

### BRIEF DESCRIPTION OF DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified schematic of a navigation apparatus which comprises a balloon catheter, an attachment mechanism, and an attached endoscope, according to an embodiment of the present invention;

FIGS. 2-6 are simplified schematics showing an exemplary method for anchoring a balloon catheter within a body conduit, according to an embodiment of the present invention;

FIGS. 7-12 are simplified schematics showing a process whereby a shaft of balloon catheter is used to guide displacement of an additional surgical tool within a body conduit or body cavity, according to an embodiment of the present invention;

FIG. 13 is a simplified schematic of an apparatus comprising a pair of balloon catheters, according to an embodiment of the present invention;

FIG. 14 is a simplified schematic of an alternative construction of an embodiment of an apparatus comprising a pair of balloon catheters, according to an embodiment of the present invention; and

FIG. 15 is a simplified schematic presenting additional optional features of an apparatus according to an embodiment of the present invention.

### DESCRIPTION OF THE PREFERRED EMBODIMENTS

The following describes apparatus and methods for navigating surgical tools within a body cavity. The invention may be used to provide easy and repeatable advancement and retraction of a surgical tool such as an endoscope within a body conduit such as an intestine.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

For exemplary purposes, the present invention is principally described in the following with reference to an exemplary context, namely that of placement and displacement of surgical tools within an intestine. It is to be understood that the invention is not limited to this exemplary context. The invention is, in general, relevant to devices and methods for navigation of one or more surgical or diagnostic tools within a body cavity, and in particular within a body conduit. For simplicity of exposition, the descriptions of embodiments provided hereinbelow, and the accompanying Figures, refer to the exemplary context of tool navigation within an intestine, yet all references to the intestinal context are to be understood to be exemplary and not limiting, and references thereto, except when referring to a specific anatomical feature such as the Ileo-cecal valve, should be understood to be generalizable to other body conduits and to natural or man-made body cavities in general.

In the following description of embodiments of the invention, the term “balloon” has been used to describe features of various embodiments as shown in various Figures. It is intended that the term “balloon” be understood in a most general sense as any expandable structure. Thus the term “balloon” as used herein, in disclosure and claims, should be understood to refer not only to an expandable flexible structure inflatable by infusion of a pressurized fluid, but also to any other expandable structure which may be provided in the context described and which would serve the function of inhibiting movement of a catheter, to which the expanding structure is attached, within a body cavity or body conduit. Thus, for example, a slotted tube expanded by advancing an object within the tube towards its tip would be a “balloon” in the context of the present disclosure and claims, as would an expanding spring controlled by a wire. A balloon may comprise additional features: for example, a balloon may or may not be transparent, may comprise an internal light source or camera, etc.

It is expected that during the life of this patent many relevant forms of catheters and many relevant forms of endoscope will be developed. The scope of the terms “catheter” and “balloon catheter” and “endoscope” are intended to include all such new technologies a priori.

In discussion of the various figures described hereinbelow, like numbers generally refer to like parts. The drawings are generally not to scale. Some optional parts, and parts not essential for understanding the invention, may be omitted.

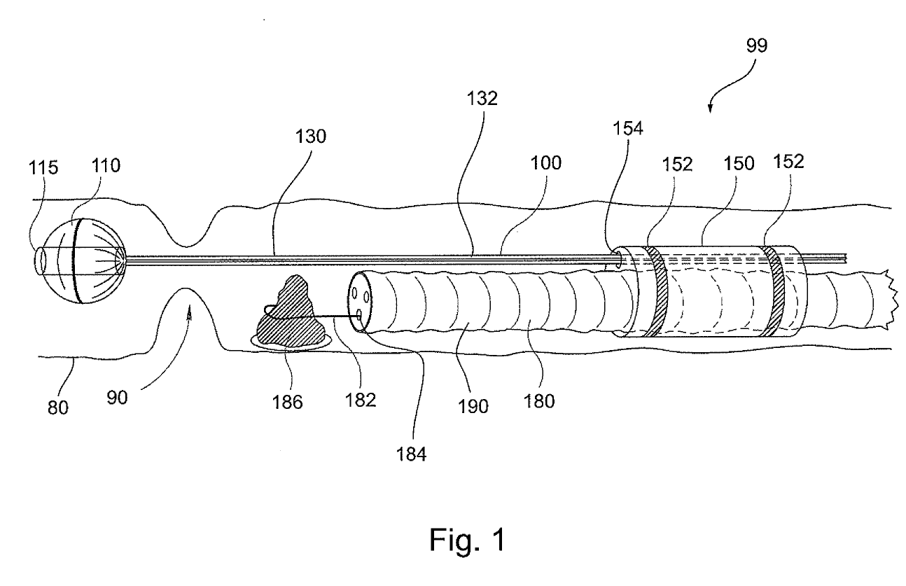

Attention is now drawn to FIG. 1, which is a simplified schematic of a navigation apparatus 99 which comprises a balloon catheter 100, an attachment mechanism 150, and an attached surgical tool 190, according to an embodiment of the present invention.

FIG. 1 presents a balloon catheter 100 having a distal head 115 which comprises an inflatable balloon 110. Catheter 100 also comprises a proximal shaft 130 which comprises an inflation lumen 132 through which a fluid may be pumped under pressure to inflate (or deflate) balloon 110. Attachment mechanism 150 is constructed so as to slidably or rigidly attach to shaft 130 and to slidably or rigidly attach to an additional tool 190, which in an exemplary embodiment shown in FIG. 1 is an endoscope 180. In the exemplary embodiment shown in FIG. 1, attachment mechanism 150 is provided with a lumen 154 sized to enable shaft 130 to slide therethrough, thereby slidably attaching catheter 110 to attachment mechanism 150, and attachment mechanism 150 is further provided with attaching bands 152 for rigidly attaching attachment mechanism 150 to a surgical tool 190, here embodied as an endoscope 180. Endoscope 180 is shown in the Figure as having an ablating tool 182 extending from a working channel of endoscope 180 to ablate a polyp 186. Balloon 110 is shown as extending beyond a narrowing 90 of a body conduit or cavity 80, where it has been inflated, thereby anchoring catheter 100 in place. Tool 190 (endoscope 180) is free to slide along shaft 130 of catheter 100 within conduit 80. A braking device or locking mechanism (not shown in the figure) may be provided to inhibit sliding as desired. For example, a wire-actuated braking mechanism that works by radial compression of the channel or radial expansion of catheter might be provided.

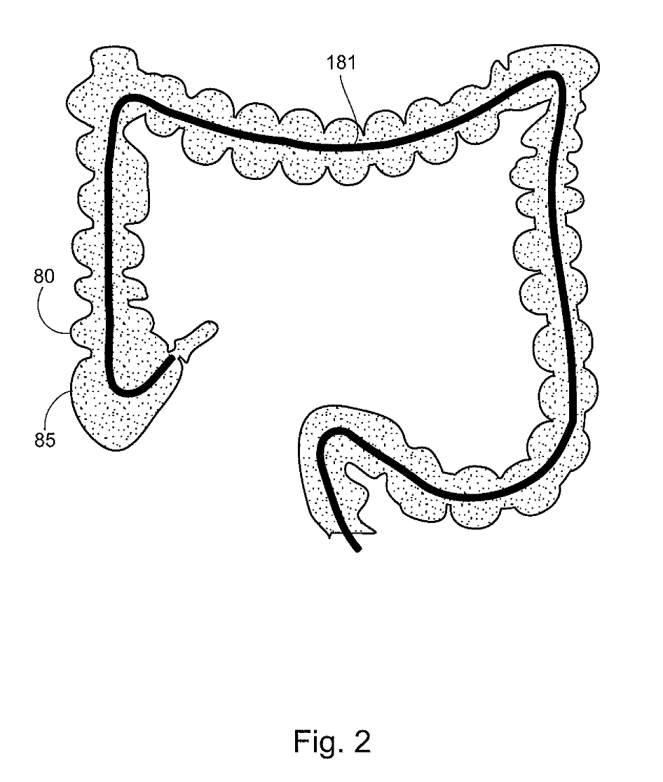

Attention is now drawn to FIGS. 2, 3, 4, 5, and 6, which are simplified schematics showing an exemplary method for anchoring catheter 100 within a body conduit, according to an embodiment of the present invention. In the exemplary context shown in FIGS. 2-6 and other Figures herein, body cavity 80 is an intestine 85. As shown in these Figures, an endoscope 181 may optionally be used to advanced catheter 100 to a narrowing of body cavity 80 or to some other desired position. Endoscope 181 may comprise an optical scope or video camera and may comprise steering mechanisms for directing the orientation of a distal portion of endoscope 181. In the exemplary context here pictured, endoscope 181 is advanced to the cecum and oriented towards the Ileo-Cecal valve. Catheter 100 is not visible in FIG. 2 because in FIG. 2 catheter 100 it to be thought of as contained within a working channel of endoscope 181.

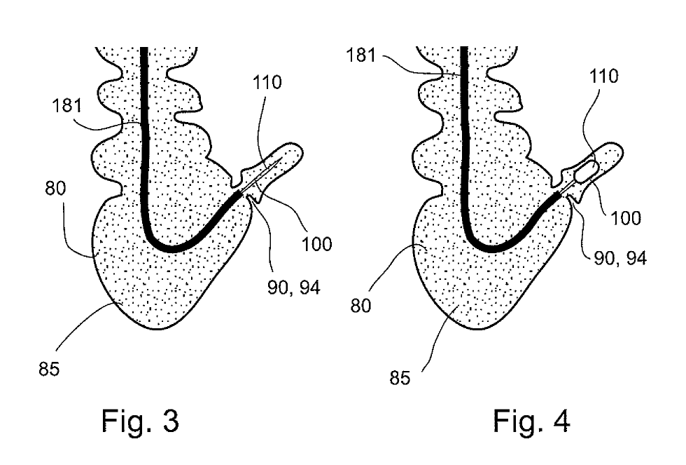

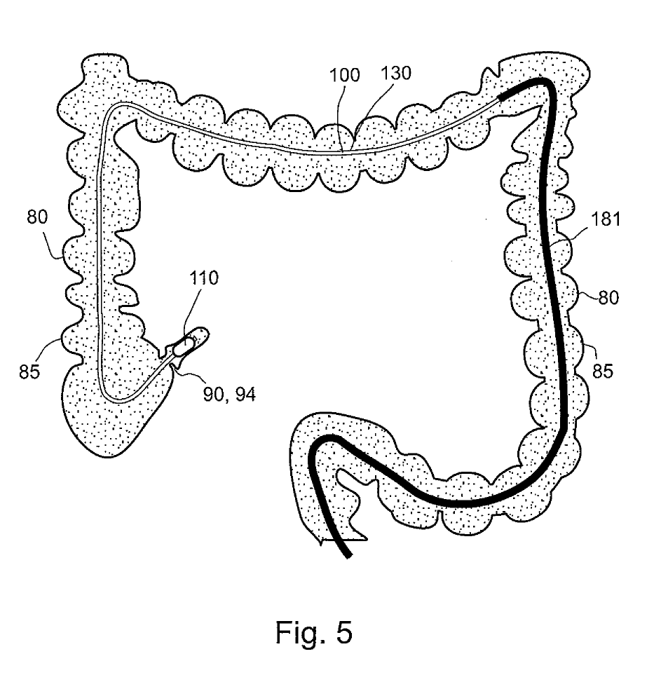

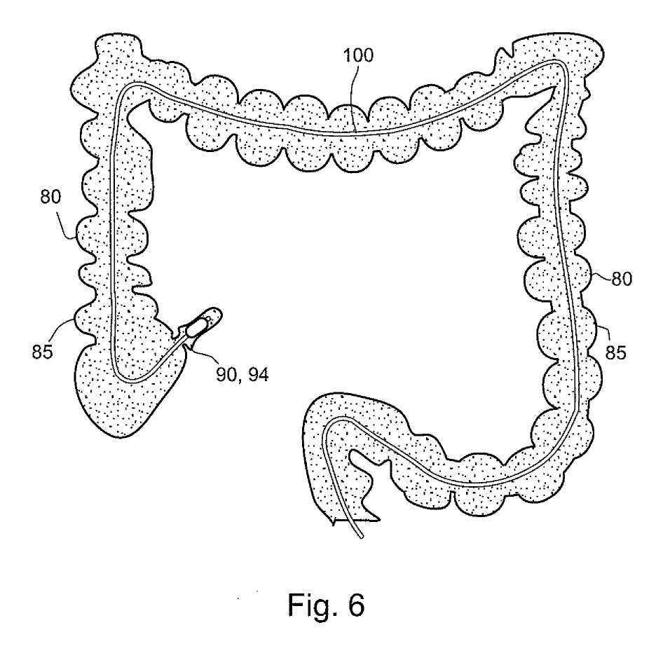

Once endoscope 181 is positioned as shown in FIG. 3, a distal portion 115 of catheter 100 may be extended from endoscope 181 to pass through and beyond the Ileo-Cecal valve 94 (or other narrowing 90 of cavity 80), and there, balloon 110 within distal portion 115 may be inflated as shown in FIG. 4. Endoscope 181 may then be pulled away from balloon 110 and out of intestine 85 (or other body conduit or body cavity 80), leaving behind catheter 100 in a position fixed by expansion of balloon 110. FIG. 5 shows a distal end of catheter 181 in an intermediate position, somewhat distanced from balloon 110 and at a selected position within intestine 85 (or cavity 80). FIG. 6 shows catheter 181 entirely removed from intestine 85 (or other body cavity or conduit 80), leaving balloon 110 of catheter 100 fixed in place and shaft 130 of catheter 100 running the length of intestine 85 (conduit/cavity 80).

It is noted that positioning balloon 110 beyond a narrowing 90 such as the Ileo-Cecal valve 94 is a convenient but not a necessary option: balloon 110 may be fixed in position in any appropriate physical context by being inflated to the point where pressure-induced friction between balloon 110 and surrounding tissue prevents or tends to prevent movement of balloon 110 from a fixed position. In some embodiments, the inflated size of balloon 110 is designed to correspond to a size appropriate for immobilizing balloon 110 within a specific context, such as within a large intestine.

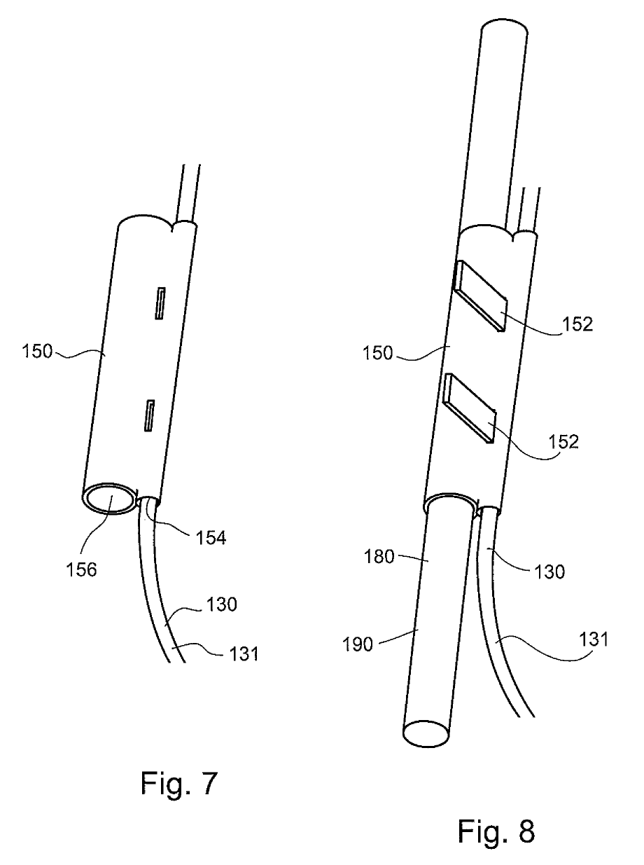

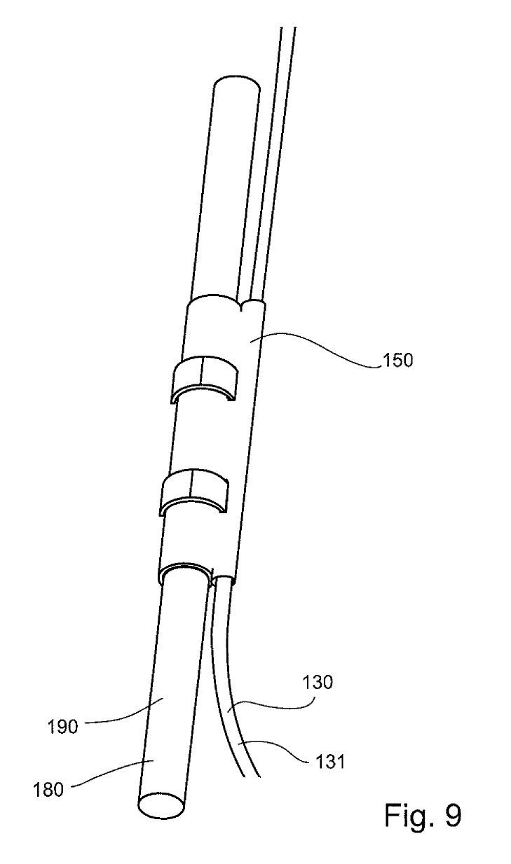

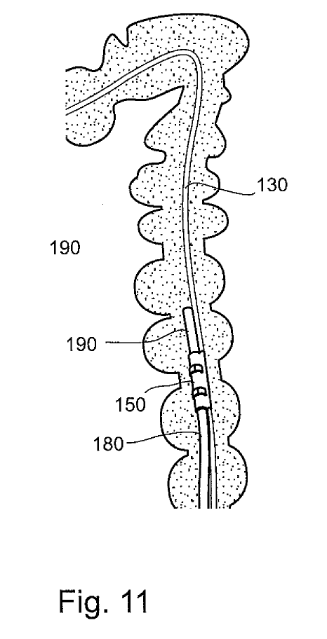

Attention is now drawn to FIGS. 7-12, which are simplified schematics showing a process whereby shaft 130 of balloon catheter 100 is used to guide displacement of an additional surgical tool within a body conduit or body cavity, according to an embodiment of the present invention.

FIG. 7 presents another exemplary embodiment of attachment mechanism 150, also presented in FIG. 1. In FIG. 7, an attachment mechanism 150 is seen to comprise a first conduit 154 for slidably accommodating shaft 130 of catheter 100. A second, optional, conduit, here labeled 156, is also provided for accommodating an additional surgical tool. In FIG. 8 an endoscope 180 is shown inserted through conduit 156 of attachment mechanism 150, shaft 130 of catheter 110 being slidably inserted through conduit 154 of attachment mechanism 150. In some embodiments attachment mechanism 150 is limited in size to 20 cm or less. In some embodiments attachment mechanism 150 is limited in size to 10 cm or less, or to 2 cm or less. In some embodiments attachment mechanism 150 may be embodied as a disposable or sterilizable sheath adapted to hold an endoscope or other surgical tool, the sheath having conduits 154 and/or 156 extending along all, or only part, of its length. Attachment mechanism 150 may be implemented as a sheath having a plurality of conduits 154 and/or 156 provided at intervals along its length.

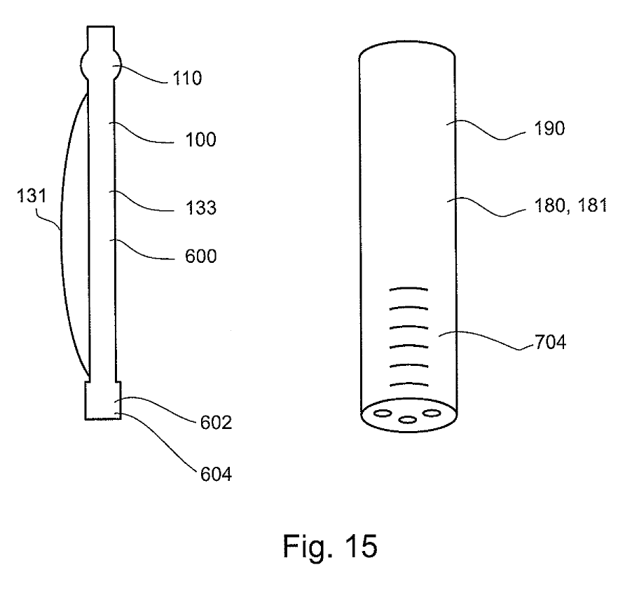

It is noted that in an alternative construction, shaft 130 of catheter 100 may be subdivided into a conduit 133 containing an inflation lumen 132 and one or more guide wires 131 (shown in FIG. 15) running substantially parallel to conduit 133 but external thereto. When this construction is used, guide wire 131 will preferably be passed through lumen 154 of attachment mechanism 150, providing a slidable mechanism for guiding attachment mechanism 150, and any tool 190 attached thereto, along shaft 130 of catheter 100.

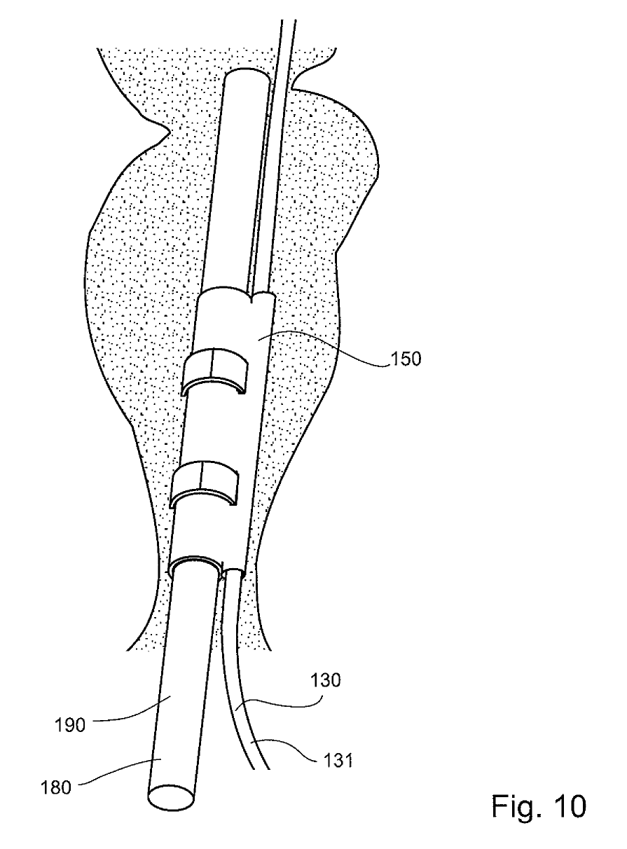

In particular embodiments of attachment mechanism 150 shown in FIG. 1 and in FIGS. 7-12, attaching bands 152 are provided for attaching tool 190 to attachment mechanism 150, as shown in particular in the sequences provided by FIGS. 7, 8, and 9. It is to be noted, however, that the particular embodiment of attachment mechanism 150 shown in these Figures is exemplary only: any attachment mechanism 150 may be used, as long as it provides and maintains a slidable connection between shaft 130 (or a portion thereof, such as a guide wire 131) and a tool 190 such as an endoscope 180 or other tool. It is further noted that attachment mechanism 150 is presented as an independent module in FIGS. 7-12, yet this presentation is not to be taken as limiting: attachment mechanism 150 may indeed be a feature incorporated within catheter 100 or within a tool 190. For example, catheter 100 and in particular shaft 130 of catheter 100 may be inserted through a working channel of an endoscope as discussed above with respect to endoscope 181 and presented by FIGS. 2-6. In that case, the working channel of endoscope 181 constitutes an attachment mechanism 150 as that term is used here, in that it provides for a slidable connection between a tool 190 (e.g. endoscope 181) and a shaft 130 of a balloon catheter 100. Thus, FIGS. 10, 11, and 12 show a process whereby a tool 190 rigidly attached to an attachment mechanism 150 which is slidably attached to a shaft 130 of a balloon catheter 100 may be slid along shaft 130 and thereby advanced into and along a body cavity or body conduit 80 such as an intestine 85, to a selected position within that cavity/conduit/intestine, and FIG. 5 shows a similar and alternative embodiment where endoscope 181 can be slid along shaft 130, with shaft 130 traversing a working channel of endoscope 181, until endoscope 181 is positioned at a desired position within the body cavity or conduit, such as an intestine. In this case, a working channel of endoscope 181 provides the functionality otherwise provided by an independent attachment mechanism 150.

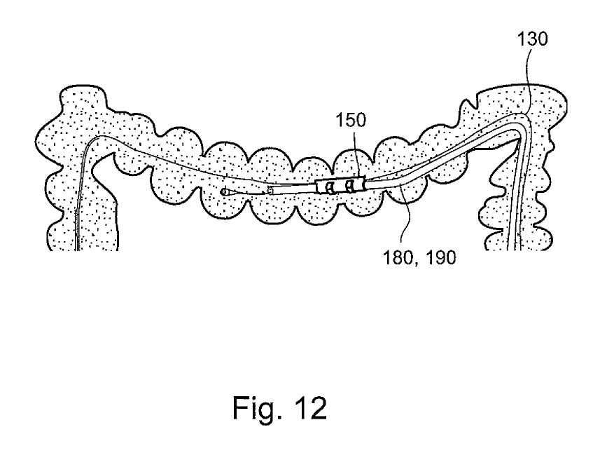

It is noted that although a single attachment mechanism 150 is shown e.g. in FIG. 12, alternatively a plurality of attachment mechanisms 150 may be provided at intervals or continuously along the length of shaft 130, for providing close slidable association between tool 190 and catheter 100 along the lengths thereof.

In a recommended mode of utilization, a tool 190 (such as an endoscope 180/181) may be provided with a shaft having visible distance markings marked thereon, the markings serving to show what length of tool 190 has been inserted within an intestine or other body conduit. The slidable association of tool 190 with shaft 130 creates a situation wherein the relationship between positioning of a distal head of tool 190 and distance markings visible on a proximal end of tool 190 (e.g. markings showing what length of an endoscope 180 has been inserted into a rectum), are highly reproducible, it is possible to position tool 190 at a selected first position, withdraw it from that position, and then easily reintroduce it to the body cavity at a new position identical to, or at least very close to, that selected first position.

This ability to rapidly and easily navigate tool 190 to a pre-selected position enables a recommended surgical technique which could not a easily be practiced without the help of apparatus 99. According to prior art surgical techniques, procedures for removal of a large growth, such as a large polyp in an intestine, involve a painstaking process whereby small portions of the polyp are excised and removed through a relatively narrow working channel of an endoscope, a time-consuming process requiring a certain number of repetitions to complete the excision of a large polyp. In contrast, apparatus 99 enables relatively rapid excision of a large polyp, because an endoscope 180 or other tool 190 can be used to excise and grasp a relatively large portion of excised tissue, a portion too large to pass easily through an endoscope working channel, and that relatively large tissue portion can be removed from the body by being grasped by tool 190 while tool 190 is removed from the body, slidably drawn backwards along shaft 130 until it exits the body. Then, using techniques described hereinabove, that tool 190 can easily and rapidly be repositioned at the excision site, to inspect the side or to practices an additional excision or to continue a systematic inspection of the intestine or other body conduit. It is noted that use of the above method enables to perform a surgery using an endoscope of smaller diameter than would otherwise be required, because removal and replacement of the endoscope can replace the need for working through an operating channel of the endoscope, consequently required operating channels can be fewer, or smaller, or absent. It is also noted that the tool removed and replaced within the body conduit may be an endoscope, or an additional tool used alongside and together with an endoscope, with that additional tool, and not the endoscope itself, being displaced and replaced as described above.

It is contemplated that the apparatus and methods described herein may be used to guide a plurality of surgical tools along a common catheter shaft. It is further contemplated that a catheter shaft may be sub-divided into a plurality of sub-shafts, each used to guide a tool 190. For example, balloon catheter 100 may be provided with a plurality of guide wires 131 enabling to attach a plurality of tools 190 each to a separate guide wire. (Guide wire 131 is seen in FIG. 15.)

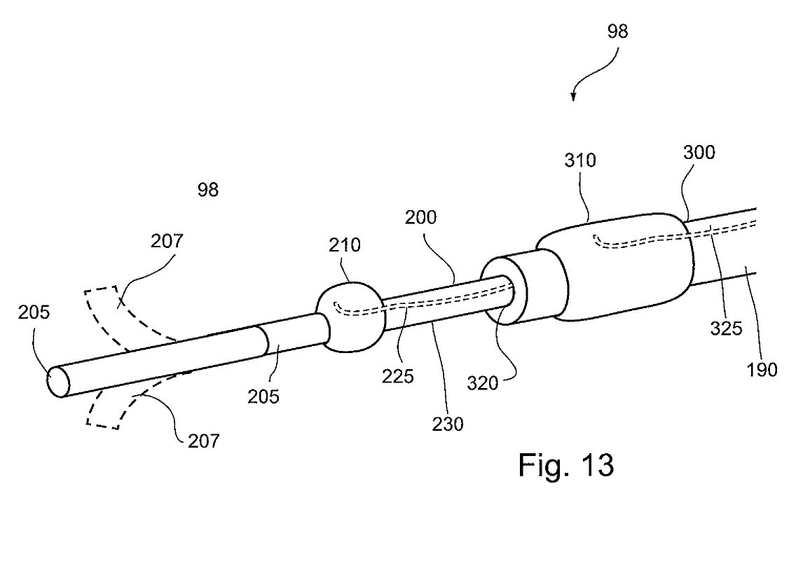

Attention is now drawn to FIG. 13, which is a simplified schematic of an apparatus comprising a pair of balloon catheters, according to an embodiment of the present invention. FIG. 13 presents an apparatus 98 which comprises a first balloon catheter 200 insertable and slidably advancable and retractable within a lumen 320 of a second balloon catheter 300. First catheter 200 comprises a balloon 210 inflatable by fluid supplied under pressure through inflation lumen 225 within shaft 230 of catheter 200. Second catheter 300 comprises a balloon 310 inflatable by fluid supplied under pressure through inflation lumen 325 within shaft 330 of catheter 300.

Apparatus 98 is a variant of apparatus 99, in that it comprises a first balloon catheter 200 with shaft 230, and a surgical tool, in this case second catheter 300, operable to slidably advance and retract along shaft 230. Either or both catheter 200 and catheter 300 can comprise or be attached to additional surgical tools such as cameras and manipulating, cutting, grasping, freezing and burning tools. Also, one or more attachment mechanisms 150 may be attached to either catheter and used to guide displacement and positioning of yet another surgical tool, using methods and devices described in detail hereinabove. Thus apparatus 98 is useful for navigating a surgical tool within a body cavity, and particularly for navigating a surgical tool within a body conduit such as an intestine.

According to an exemplary method of use, catheter 200 is first advanced into a body conduit such as an intestine for a first convenient distance. Balloon 210 is there inflated, fixing catheter 200 at that position within the conduit. Catheter 300 is then pushed forward along shaft 230 a desired distance. Balloon 310 is then inflated, fixing catheter 300 at its position within the conduit. Balloon 210 is then deflated, freeing catheter 200 for further movement. Catheter 200 is then further advanced by being pushed forward from its proximal end. Since catheter 200 is supported along part or most of its length by catheter 300, which is now in a fixed position, catheter 200 is easily advanced within the conduit. Alternatively, the order of these operations may be reversed, with catheter 300 first advanced, then catheter 200, then again catheter 300, etc. In either case the process may be repeated as needed, advancing the pair of catheters into the target body conduit in successive steps.

Catheter 200, and optionally both catheters, may be constructed with a relatively flexible distal portion, enabling them to follow the twists and turns of a body conduit, and a relatively stiff proximal portion, facilitating being advanced by being pushed forward from outside the body. Distal head 205 of catheter 200 may be provided with an aiming mechanism (internal to 205, effect shown as 207) enabling to orient head 205 in selected directions.

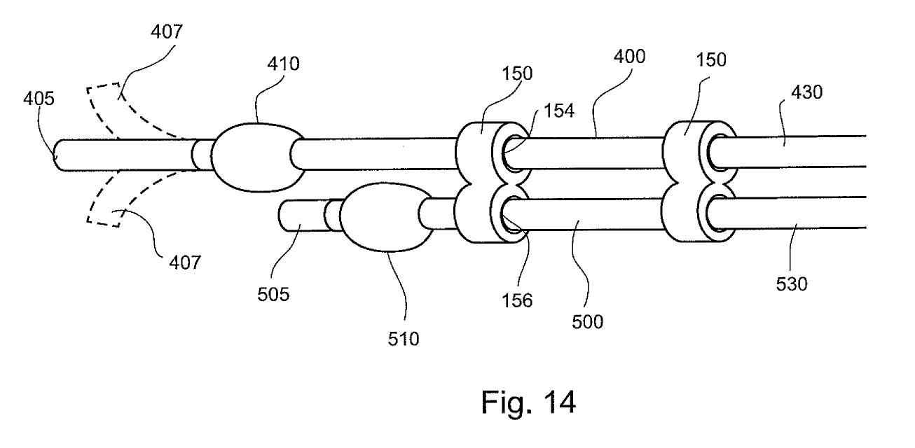

Attention is now drawn to FIG. 14, which is a simplified schematic of an alternative construction of an embodiment of an apparatus comprising a pair of balloon catheters, according to an embodiment of the present invention. FIG. 14 presents an apparatus 97 which comprises a first balloon catheter 400 insertable and slidably advancable and retractable within a first lumen 154 of an attachment apparatus 150, and a second balloon catheter 400 either slidably or fixedly inserted within a second lumen 156 of attachment apparatus 150. Attachment apparatus 150 I shown in slightly exaggeration proportions for clarity of the figure, but should be constructed to present a low profile without sharp corners, to facilitate its movement within a body conduit. First catheter 400 comprises a balloon 410 inflatable by fluid supplied under pressure through an inflation lumen (not shown) within shaft 430 of catheter 400. Second catheter 500 comprises a balloon 510 inflatable by fluid supplied under pressure through an inflation lumen within shaft 530 of catheter 500.

Apparatus 97 is an embodiment of apparatus 99, in that it comprises a first balloon catheter 400 with shaft 430, and a surgical tool, in this case second catheter 500, operable to slidably advance and retract along shaft 430. Either or both catheter 400 and catheter 500 can comprise or be attached to additional surgical tools such as cameras and manipulating, cutting, grasping, freezing and burning tools. Also, one or more additional attachment mechanisms 150 may be attached to either catheter and used to guide displacement and positioning of yet additional surgical tools, using methods and devices described in detail hereinabove. Thus apparatus 97 is useful for navigating a surgical tool within a body cavity, and particularly for navigating a surgical tool within a body conduit such as an intestine. First and second catheters of apparatus 97 may be detachably connected or permanently connected.

According to a recommended method of use, catheter 400 is first advanced into a body conduit such as an intestine for a first convenient distance. Balloon 410 is there inflated, fixing catheter 400 at that position within the conduit. Catheter 500 is then pushed forward along shaft 430 a desired distance. Balloon 510 is then inflated, fixing catheter 500 at its position within the conduit. Balloon 410 is then deflated, freeing catheter 400 for further movement. Catheter 400 is then further advanced by being pushed forward from its proximal end. Since catheter 400 is supported along part or most of its length by one or preferably a plurality of attachment mechanisms 150 slidably attaching it to catheter 500, which is now in a fixed position, catheter 400 is easily advanced within the conduit. Catheter 400, and optionally both catheters, may be constructed with a relatively flexible distal portion, enabling them to follow the twists and turns of a body conduit, and a relatively stiff proximal portion, facilitating being advanced by being pushed forward from outside the body. Distal head 405 of catheter 200 may be provided with an aiming mechanism (internal to 405, effect shown as 407) enabling to orient head 405 in selected directions. As discussed above with respect to FIG. 13, the order of operations described in this paragraph may be inversed, and the process may be repeated as needed.

Attention is now drawn to FIG. 15, which is a simplified schematic of apparatus 99 showing several optional features, according to embodiments of the present invention.

FIG. 15 presents optional guide wire 131, considered to be a part of shaft 130 of catheter 100. Use of guide wire 131 has been discussed hereinabove.

FIG. 15 also presents a removable proximal connector 602 for connecting catheter 100 to a source of pressurized fluid used for inflating balloon 110. Connector 602 may be a removable connector such as a Tuohy-Borst connector 604 or other removable connector, provided to enable to insert catheter shaft 130 into a narrow conduit such as a working channel of an endoscope, with the insertion taking place starting from the proximal end of catheter 100. FIG. 15 also presents distance markings 704 on a proximal shaft portion of a surgical tool 190 such as an endoscope 180 or 181. Use of such markings has been discussed hereinabove. It is noted that markings 704 may be provided elsewhere within the apparatus, for example in a position visible by endoscope, so a user can see the markings without his eye leaving an endoscope-generated image. Location information may also be read electronically and reproduced in a display and/or used to control automated processes for delivering tool 190 to a target locus.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. An apparatus for navigating a surgical tool within a body, comprising:
(a) a first catheter having
(i) a distal head which comprises a first expandable element; and
(ii) a proximal shaft, and
(b) a first tool operable to slide along at least a portion of the shaft.

2. The apparatus of claim 1, wherein the first expandable element is an inflatable balloon.

3. The apparatus of claim 2, wherein the first tool is slidably connected to the shaft.

4. The apparatus of claim 2, wherein the first tool comprises an attachment mechanism for connecting the first tool to the shaft.

5. The apparatus of claim 2, wherein the first tool comprises an attachment mechanism for attaching a second tool to the shaft.

6. The apparatus of claim 5, wherein the attachment mechanism is slidably connectable to the shaft and rigidly connectable to a second tool.

7. The apparatus of claim 5, wherein the attachment mechanism is a sleeve which comprises a binding for rigidly attaching the second tool to the attachment mechanism, and further comprises a lumen through which the catheter shaft may be inserted.

8. The apparatus of claim 2, wherein the first tool is an endoscope.

9. The apparatus of claim 5, wherein the second tool is an endoscope.

10. The apparatus of claim 9, wherein the first catheter, attachment mechanism, and endoscope are packaged as a kit.

11. The apparatus of claim 8, wherein the first catheter is insertable in a working channel of the endoscope.

12. The apparatus of claim 9, wherein the catheter is insertable through a conduit connectable to the endoscope.

13. The apparatus of claim 12, wherein the conduit is less than 20 cm long.

14. The apparatus of claim 13, wherein the conduit is less than 10 cm long.

15. The apparatus of claim 14, wherein the conduit is less than 2 cm long.

16. The apparatus of claim 12 wherein the conduit is comprised within a disposable sheath into which an endoscope may be inserted.

17. The apparatus of claim 5, wherein the second tool is a second catheter which comprises a second inflatable balloon.

18. The apparatus of claim 2, wherein the first tool comprises a second catheter which comprises a second inflatable balloon.

19. The apparatus of claim 18, wherein the second catheter is slidably connected to the first catheter and external thereto.

20. The apparatus of claim 19, wherein the second catheter is connectable to the first catheter by means of an attachment mechanism.

21-50. (canceled)
